(19) United States
(12) Patent Application Publication
(10) Pub. No.: US 2023/0204513 A1
(43) Pub. Date: Jun. 29, 2023

(54) Combined Fluorescence and Scanning Electron Microscope System for Pathogen Detection

(71) Applicant: IntelliFoods Labs, LLC, Weston, MA (US)

(72) Inventors: Mohammad E. Taslim, Needham, MA (US); Mehdi Abedi, Roslindale, MA (US); Ramin M. Hakami, Bristow, VA (US); Michael Mojaver, Aptos, CA (US); Reza Mollaaghababa, Natick, MA (US); Edward A. Greenfield, Stoughton, MA (US)

(73) Assignee: IntelliFoods Labs, LLC, Weston, MA (US)

(21) Appl. No.: 18/075,059

(22) Filed: Dec. 5, 2022

Related U.S. Application Data
(60) Provisional application No. 63/286,223, filed on Dec. 6, 2021.

(51) Int. Cl. G01N 21/64; G01N 23/2251

(52) U.S. Cl. CPC G01N 21/6428; G01N 23/2251; G01N 2021/6441

(57) ABSTRACT

A method of detecting a pathogen in a sample, comprising: mixing at least a portion of the sample with a plurality of capture particles functionalized with a molecular recognition element exhibiting specific binding to said pathogen to capture at least a portion of pathogen particles when present in the sample; exposing said captured pathogen particles to at least two fluorescent dyes, which emit fluorescent radiation at two different wavelengths in response to excitation, such that live pathogen particles among said captured pathogen particles are preferentially stained with one of the dyes and dead pathogen particles among said captured pathogen particles are preferentially stained with the other dye; irradiating the captured stained pathogen particles with excitation radiation to excite said fluorescent dyes; and detecting fluorescent radiation emitted by said excited fluorescent dyes; and distinguishing said live pathogen particles from said dead pathogen particles based on wavelengths of the detected fluorescent radiation.

### RELATED APPLICATIONS

This patent application claims the benefit of U.S. Provisional Application No. 63/286,223, filed on Dec. 6, 2021, entitled “A Combined Fluorescence and Scanning Electron Microscope System for Pathogen Detection,” the contents and teachings of which are hereby incorporated by reference in their entirety.

### TECHNICAL FIELD

The present teachings are generally directed to methods and systems for detecting pathogens in samples, such as food samples.

### BACKGROUND

Pathogens, ubiquitous in nature, can be major vectors of infection around the globe, and can cause a variety of infectious diseases. The detection of pathogens is thus of significant importance in a variety of domains. For example, in the agricultural and food industry, it is crucial to have sensitive and accurate technologies for the detection of a variety of food-borne pathogens to ensure that the food supply is safe.

### SUMMARY

Conventional techniques for detecting food-borne pathogens in food samples typically suffer from certain limitations. For example, the gold standard for testing food samples is the polymerase chain reaction (PCR), such as real-time quantitative PCR (qPCR). Although PCR techniques are highly accurate and sensitive, they can be time consuming, which can result in a significant time delay (e.g., in a range of 24 to 48 hours) in delivering food supply to consumers. Such a time delay may result in undesirable alterations in taste, smell, appearance and/or texture of the food items, and even lead to spoilage of the food items before their delivery to consumers.

Accordingly, there is a need for improved methods and systems for detecting pathogens in a variety of samples.

In one aspect, a method of detecting a pathogen in a sample, e.g., a food sample, is disclosed, which comprises mixing at least a portion of the sample with a plurality of capture particles functionalized with a molecular recognition element exhibiting specific binding to said pathogen so as to capture at least a portion of pathogen particles when present in the sample, and exposing the captured pathogen particles to at least two fluorescent dyes, which emit fluorescent radiation at two different wavelengths in response to excitation, such that live pathogen particles, if any, among the captured pathogen particles are preferentially stained with one of the dyes and dead pathogen particles, if any, among the captured pathogen particles are preferentially stained with the other dye. The captured stained pathogen particles are then irradiated with an excitation radiation so as to excite the fluorescent dyes, and fluorescent radiation emitted by the fluorescent dyes in response to such excitation is detected and analyzed to distinguish the live pathogen particles from the dead pathogen particles, if present in the sample under investigation.

In particular, the live pathogen particles and the dead pathogen particles can be distinguished from one another based on the wavelengths of the detected fluorescent radiation. The analysis of the emitted fluorescent radiation can be based on the wavelengths of the detected fluorescent radiation. By way of example, the detection of the emitted fluorescent radiation in the channel associated with the dye that preferentially stains the live pathogen particles can indicate the presence of the live pathogen in the sample under investigation and the detection of the emitted fluorescent radiation in the channel associated with the dye that preferentially stains the dead pathogen particles can indicate the presence of the dead pathogen in the sample. The detection of emitted fluorescent radiation in both fluorescent channels can indicate the presence of both live and dead pathogens in the sample.

The present teachings can be applied for analysis of a variety of different samples. By way of example, the sample under investigation can be a food sample. In such embodiments, the pathogen can be a food-borne pathogen. Such food-borne pathogens can be, for example, E. coli, Salmonella, Listeria, Clostridium, Staphlococcus, Campylobacter and Shigella bacteria. In some embodiments, the present teachings can be applied to detect fungi, e.g., Cyclopora, and viruses, such as Norovirus, Vibrio, Toxoplasmosis and Hepatitis A, among others.

In some embodiment, the capture particles can be magnetic particles that are functionalized with the molecular recognition element. In some such embodiments, a magnetic field can be applied to the magnetic particles so as to concentrate the magnetic particles coupled to the pathogen particles, if any. In some such embodiments, the step of irradiating the captured stained pathogen particles with the excitation radiation is performed subsequent to the step of applying the magnetic field to cause concentration of the magnetic particles coupled to the pathogen particles.

In some embodiments, the magnetic particles coupled to the pathogen particles are introduced into a scanning electron microscope (SEM) and one or more SEM images of at least a portion of the magnetic particles are acquired. In some embodiments, the introduction of the pathogen-coupled magnetic particles into the SEM is performed after staining the pathogen particles, if any, present in the sample.

One or more of the SEM images can be analyzed to identify one or more pathogen particles, if any, that are coupled to the magnetic particles. In some such embodiments, the analysis of the SEM images can be performed using artificial intelligent techniques.

In some embodiments, the detected fluorescent radiation can be processed to generate one or more fluorescent images of the pathogen-coupled magnetic particles. In some embodiments, such a fluorescent image and a respective SEM image are correlated to identify a one-to-one correspondence between the fluorescent images of one or more pathogen particles detected in the fluorescent image(s) with the respective SEM images of those pathogens. In some embodiments, such a correlation can be useful in increasing the confidence in the detection of a pathogen in the sample.

A variety of molecular recognition elements can be employed in the practice of the present teachings. By way of example, some examples of suitable molecular recognition elements can include an antibody or an aptamer among others.

In a related aspect, a system for detecting at least one pathogen in a sample is disclosed, which comprises a substrate, a plurality of capture particles each functionalized with at least one molecular recognition element for specifically binding to said at least one pathogen, said plurality of capture particles being configured for distribution over a surface of said substrate. The system can further include a scanning electron microscope having a holder for receiving the substrate, where the system is configured for generating one more electron microscopy images for the plurality of particles disposed on said substrate surface. An artificial analysis module can receive one or more of the SEM images and analyze those images for the detection of one or more of the pathogen particles, if any, bound to one or more of the capture particles.

In some embodiments of the above system, the capture particles include a plurality of magnetic particles. In some such systems, a magnet, e.g., an electromagnet, can be employed to apply a magnetic field to the magnetic particles, e.g., subsequent to mixing of the magnetic particles with a sample under investigation for capturing one or more pathogen particles, if any, within the sample, in order to facilitate distribution of the plurality of the magnetic particles onto the substrate surface.

In some embodiments, the substrate holder can be configured to be movable relative to an electron beam of the electron microscope so as to allow acquisition of the one or more electron microscope images from different viewing angles.

In some embodiments, the capture particles, e.g., a plurality of magnetic particles, can have a maximum size, e.g., a maximum diameter in cases where the capture particles are substantially spherical, equal to or less than about 2 micrometers, e.g., in a range of about 100 nm to about 2 micrometers, or in a range of about 500 nm to about 1 micron.

A variety of the molecular recognition elements can be employed, such as an antibody, an aptamer, or any other suitable molecular recognition element that exhibits specific binding to a pathogen of interest.

In some embodiments, the capture particles can include particles with different sizes. For example, the plurality of capture particles can include a first set of particles having a first size (e.g., a first diameter) and a second set of particles having a second size (e.g., a second diameter) different than the first size. In some such embodiments, the first set of the capture particles can be functionalized with a first molecular recognition element exhibiting specific binding to a first pathogen and the second set of particles can be functionalized with a second molecular recognition element exhibiting specific binding to a second different pathogen.

In some embodiments, the system can include at least one sample-processing reagent for processing the sample prior to exposing the sample to the fluorescent dyes and/or mixing the sample with the capture particles, which are functionalized with a suitable molecular recognition element.

A system according to the present teachings can be employed for analysis of a plurality of samples. For example, such a system can be employed to analyze a food sample for detecting pathogens of interest, e.g., food-borne pathogens, in the sample.

In a related aspect, a method of detecting at least one pathogen in a sample is disclosed, which comprises processing a sample with one or more processing reagents to generate a processed sample and mixing the processed sample with a plurality of capture particles functionalized with at least one molecular recognition element exhibiting specific binding to said at least one pathogen. One or more electron microscopy images of said plurality of the particles are generated and an artificial intelligence system is employed to identify the image(s) of pathogen particles in said one or more microscopy images, when the pathogen particles are present in the sample.

In some embodiments of the above method, subsequent to the mixing step, at least a portion of the plurality of the capture particles is introduced onto a surface of a substrate. By way of example, the substrate can be a semiconductor substrate or a polymeric substrate, among others. The substrate can then be introduced into the SEM. A magnetic field can be utilized to facilitate the introduction of the magnetic particles onto the surface of the substrate.

In some embodiments, a system according to the present teachings can include a module for processing a sample, an imaging module and/or an analysis module. In some embodiments, the sample-processing module can receive a substrate and a sample for analysis. The sample-processing module can be configured to prepare the sample, including mixing the sample with a plurality of functionalized magnetic particles and deposit the magnetic particles onto a substrate surface (e.g., a semiconductor substrate). The system can further include a mechanism for automatically transferring the substrate from the sample-processing module into the fluorescence/SEM imaging module. Such a transfer mechanism may include, for example, a carriage to which the substrate can be coupled, where the carriage can move along a pair of rails to transfer the substrate from sample-processing module to the fluorescence/SEM imaging module. The system can further include an analysis module that is implemented in hardware, firmware and/or software. In some embodiments, the analysis module can include an AI engine that is configured to analyze the fluorescent and/or SEM images to identify image(s) of pathogen particles, if any, in those images. For example, in some embodiments, supervised or unsupervised algorithms can be employed to process the fluorescent and/or SEM images. The analysis module can be further configured to provide a notification regarding the presence or absence of the pathogen of interest in the sample under investigation, e.g., based on the output of the AI engine.

As noted above, the methods and systems according to the present teachings can be employed to detect a variety of pathogens in a variety of different samples, such as food and environmental samples (e.g., water samples). Some examples of pathogens can be detected using the methods and systems according to the present teachings include, without limitation, Campylobacter, Clostridium perfringens, Norovirus, Bacillus cereus, Botulism, Hepatitis A, Shigella, Staphylococcus aureus (Staphylococcal [Staph] Food Poisoning), and Vibrio Species Causing Vibriosis. In fact, any pathogen for which a suitable molecular recognition element is available and/or can be generated can be detected in accordance with the present teachings, e.g., via using capture particles that are functionalized with that molecular recognition element.

Further understanding of various aspects of the present teachings can be obtained by reference to the following detailed description in conjunction with the associated drawings, which are described briefly below.

### BRIEF DESCRIPTION OF THE DRAWINGS

FIG. 1 is a flow chart depicting various steps in a method according to the present teachings for detecting pathogen particles in a food sample,

FIG. 2 schematically depicts a system according to an embodiment of the present teachings for detecting pathogen particles in a food sample,

FIGS. 3A-3E schematically show various stages in an implementation of a method according to an embodiment of the present teachings for detecting pathogen particles in a food sample,

FIG. 3F schematically depicts four modules of a system according to an embodiment, including a sample pre-processing unit, a sample preparation unit, an imaging unit, and AI post-processing unit.

FIG. 4 schematically depicts a sample processing module suitable for use in embodiments of systems according to the present teachings for detecting pathogen particles in a food sample,

FIG. 5 schematically depicts a system according to an embodiment of the present teachings for detecting pathogen particles in a food sample, which includes the food processing module shown in FIG. 4 as well as a food sample analysis module, which includes a fluorescent and an SEM imaging system,

FIG. 6 schematically depicts an example of sectional a fluorescent imaging system suitable for use in the pathogen-detection system depicted in FIG. 5,

FIG. 7 schematically depicts an example side sectional view of an SEM imaging system suitable for use in the pathogen-detection system depicted in FIG. 5,

FIG. 8 shows an SEM image of a plurality of functionalized magnetic particles to some of which E. coli bacteria are attached,

FIGS. 9A and 9B show fluorescent images of stained E. coli bacteria,

FIG. 10 is a graph illustrating the results of binding of E. coli bacteria to beads functionalized with an antibody,

FIG. 11 is a graph illustrating the results of binding of E. coli bacteria to beads functionalized with a different antibody, and

FIG. 12 is a schematic depiction of two types of capture particles and having different sizes.

### DETAILED DESCRIPTION

The present teachings are generally related to methods and systems for detecting pathogens in a sample, such as a food sample or an environmental sample.

Various terms are used in accordance with their ordinary meanings in the art unless otherwise indicated. The term “antibody,” as used herein, refers to macromolecules typically comprising two large heavy chains and two small light chains, as well as fragments of such macromolecules (e.g., fragment antigen-binding (Fab) and fragment crystallization (Fc)) and recombinant proteins containing antibody fragments (e.g., antigen-binding portion).

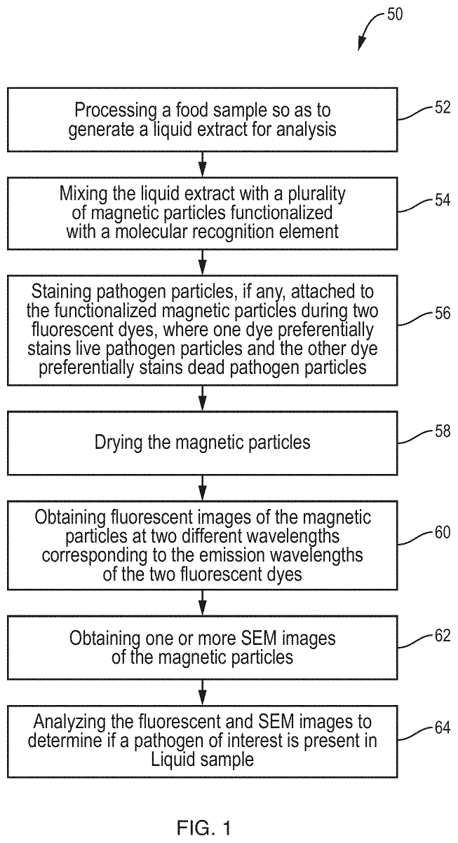

With reference to the flow chart 50 of FIG. 1, an embodiment of a method according to the present teachings for detecting pathogens in a food sample includes an initial step of processing a food sample so as to extract a liquid extract for analysis 52. Such processing of the food sample can include, for example, mixing the food sample with one or more processing liquids (e.g., a mixture of water and PBS (phosphate buffered saline) and blending the mixture followed by filtering to obtain a liquid extract.

As provided in element 54, the liquid extract can then be mixed with a plurality of magnetic particles (e.g., ferromagnetic particles) that are functionalized with at least one molecular recognition element (e.g., an antibody) that exhibits specific binding to a pathogen of interest to generate a liquid sample. In some cases, the liquid extract can be mixed with two or more populations of magnetic particles that have different sizes and are functionalized with different molecular recognition elements that provide specific binding to different pathogens. By way of example, a portion of the magnetic particles can be functionalized with an antibody that exhibits specific binding to E. coli bacterium and another portion of the magnetic particles can be functionalized with an antibody that exhibits specific binding to Salmonella bacterium. Further, the sizes of the two populations of the magnetic particles can be different to allow distinguishing between the two pathogens in SEM images of the functionalized magnetic particles coupled to the magnetic particles, as discussed in more detail below. The mixing of the liquid extract with the functionalized magnetic particles can result in the attachment of at least a portion of the pathogen particles of interest to the functionalized magnetic particles via binding to the molecular recognition element.

As indicated by element 56, the liquid sample can then be mixed with a liquid containing two fluorescent dyes, one of which preferentially stains live pathogen particles and the other preferentially stains dead pathogen particles, if any, attached to the functionalized magnetic particles.

In some embodiments, multiple cycles of staining followed by washing can be performed and subsequently, the functionalized magnetic particles with the associated stained pathogen particles, if any, can be dried, e.g., using an inert drying gas (such as nitrogen).

Next, the magnetic particles can be dried, as indicated in element 58. As indicated in element 60 fluorescent images of the functionalized magnetic particles can then be acquired at two different wavelengths corresponding to the emission wavelengths of the two fluorescent dyes. As indicated in element 62, one or more SEM images of the magnetic particles can be obtained and analyzed to identify pathogen particles, if any, in the SEM image. In some embodiments, the systems and methods according to the present teachings allow the detection of a pathogen in the liquid sample at a concentration as low as 1 CFU (colony-forming unit) per 100 milliliters.

For example, the magnetic particles can be irradiated with excitation radiation suitable for exciting the fluorescent dyes and fluorescent emission, if any, emanating from the sample can be detected. The detection of the fluorescent emission from the sample in response to excitation can be indicative of the presence of pathogen particles in the liquid sample. Further, as noted above, the wavelength of the fluorescent emission can be used to distinguish between live and dead pathogen particles.

As indicated in element 64, the fluorescent and SEM image(s) can then be analyzed to identify pathogen particles, if any, attached to the magnetic particles and present in the liquid samples. In some implementations, artificial intelligence (AI) techniques may be utilized for processing the SEM image(s) so as to identify pathogen(s) of interest, if any, within the image. For example, an AI system can be trained using images of functionalized magnetic particles attached to a pathogen of interest. The trained AI system can then be used to process the acquired SEM images to identify the image(s) of the pathogen particles, if any, within those images.

In some embodiments, the SEM and the fluorescent images can be correlated to define a one-to-one correspondence between each pathogen particle identified within both images. This can advantageously increase the confidence level associated with the detection of the pathogen particles. In some embodiments, a holder within the SEM module that is configured for holding the substrate can be movable so as to allow changing the angle at which the magnetic particles are exposed to the electron beam, thereby allowing the acquisition of SEM images from different angles. This can help identify pathogen particles, if any, that may not be present in one anguview but visible in another angular view. For example, in a default mode, the scanning electron beam can be directed onto the substrate in a direction orthogonal to the substrate.

In some cases, a pathogen particle identified within a fluorescent image may not be detected in a respective SEM image, or vice versa. In some embodiments, such an outcome may be designated as being inconclusive, and may require testing another portion of the sample.

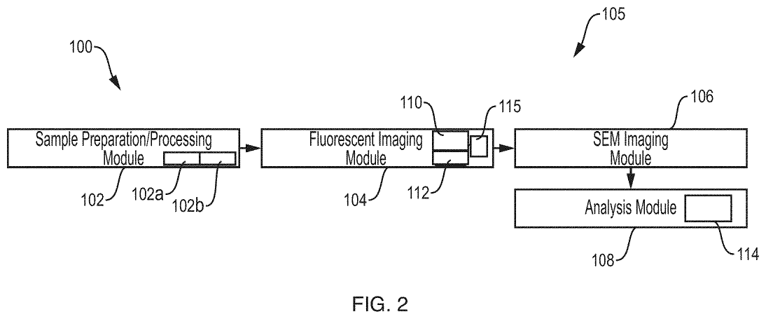

FIG. 2 schematically depicts a system 100 according to an embodiment for detecting pathogens in a sample, which includes a sample-preparation and processing module 102, a combined fluorescent/SEM imaging system 105 that includes a fluorescent imaging module 104 and an SEM imaging module 106, and an analysis module 108.

The sample-preparation/processing module 102 can receive a sample (e.g., a food sample) and generate a liquid sample from the food sample, e.g., in a manner discussed above.

The fluorescent imaging module 104 can receive the liquid sample and can include one or more radiation sources generating excitation radiation suitable for exciting the fluorescent dyes staining pathogen particles, if any, that are attached to the functionalized magnetic particles. The fluorescent imaging module 104 can further include at least one detector 110 for detecting the fluorescent radiation emitted by the excited fluorescent dyes, if any. In some embodiments, the fluorescent imaging module 104 can include two detectors 110, 112 with appropriate filters, such that one detector is dedicated to detecting the emitted fluorescent radiation in one of the fluorescent channels associated with one of the dyes and the other detector is dedicated to detecting the emitted fluorescent radiation in the other fluorescent channel associated with the other dye.

The fluorescent imaging module 104 can further include an analysis module 115 that can receive fluorescent detection signals generated by the detector(s) 110 in response to the detection of emitted fluorescent radiation and can process those detection signals to determine whether any live and/or dead pathogen particles of interest can be identified in the sample under analysis. In some embodiments, the analysis module 115 can be configured to process the fluorescent detection signals to provide a quantitative measure of the number of live and/or dead pathogen particles, if any, within the sample.

With continued reference to FIG. 2, the SEM imaging module 106 can receive the functionalized magnetic particles and can be employed to obtain an SEM image of the magnetic particles. The analysis module 108 can receive the image data generated by the fluorescent and SEM imaging modules and process that data to identify one or more pathogens of interest, if any, in the image data. By way of example, an AI analysis system 114 of the analysis module 108, such as a computerized device having a memory and a processor, can be configured to process the SEM images, using for example supervised AI techniques, to identify the magnetic particles and the pathogen particles coupled to the magnetic particles. In some implementations, the AI analysis system 114 can be trained to identify the type of pathogen particles from an SEM image thereof. For example, the AI analysis system 114 may identify that the pathogen particle identified in the SEM image is an E. coli or Salmonella bacteria.

As noted above, in some cases, magnetic particles of different sizes are functionalized with different molecular recognition elements exhibiting specific binding to different pathogens. This allows the identification of the pathogen type in the SEM image based on the size of the magnetic particle to which the pathogen particle is coupled.

In some embodiments, the fluorescent and SEM imaging modules 104, 106 can be integrated within the same housing and can be configured to allow sequential or concurrent acquisition of fluorescent and SEM images.

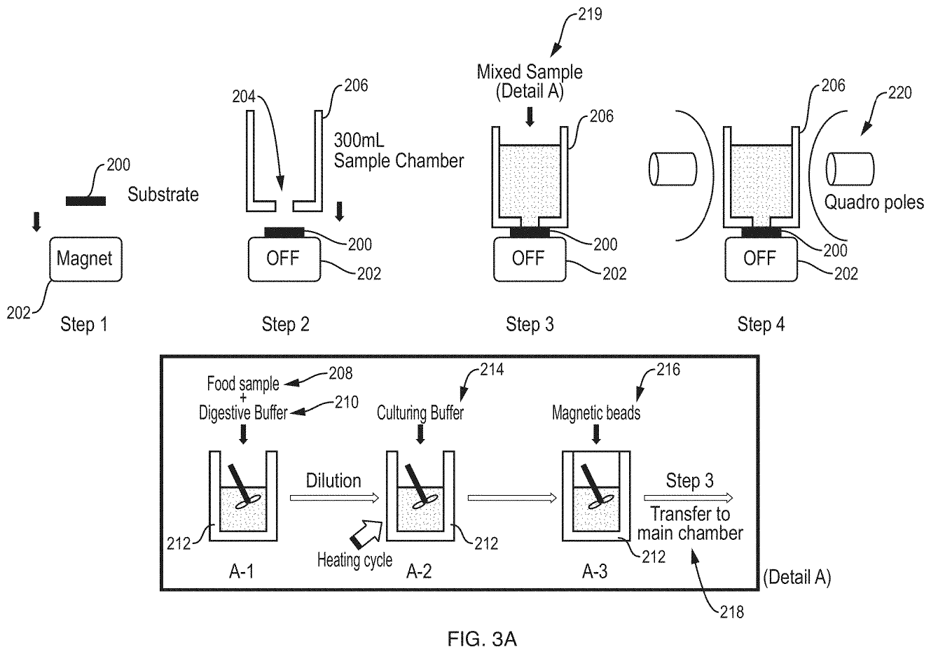

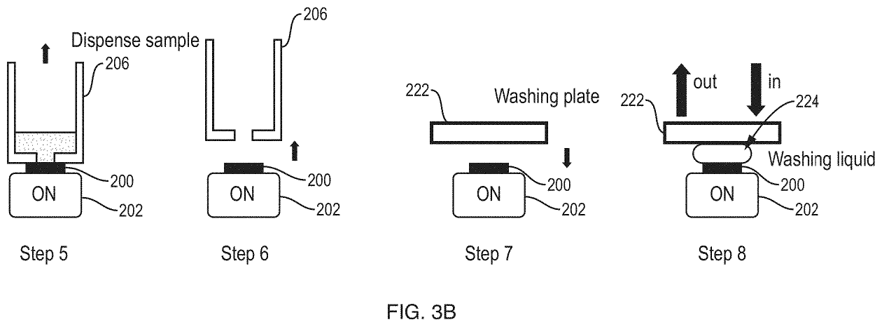

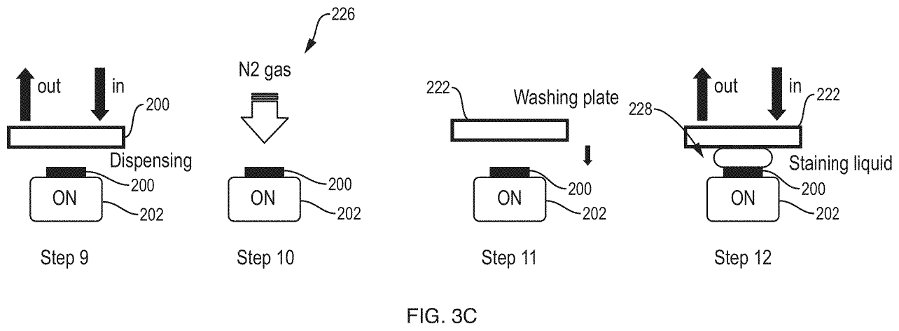

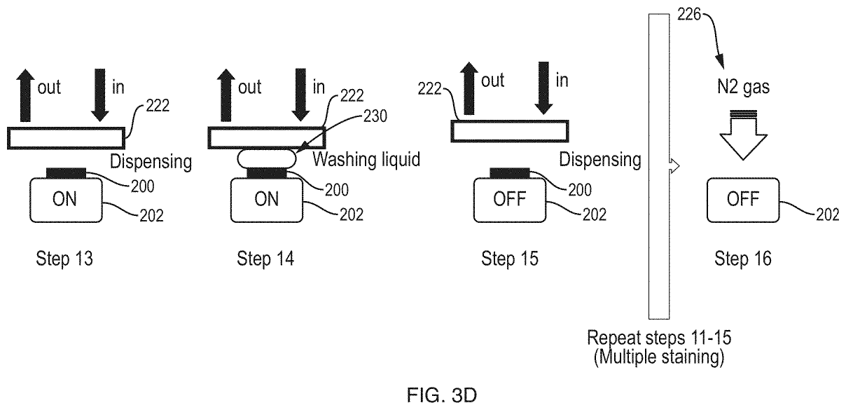

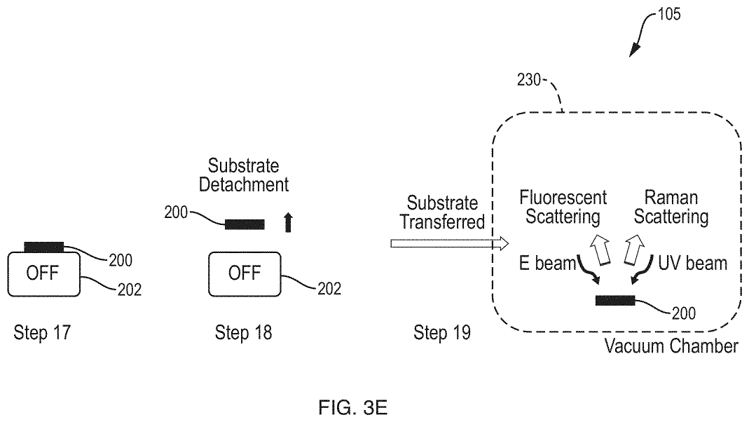

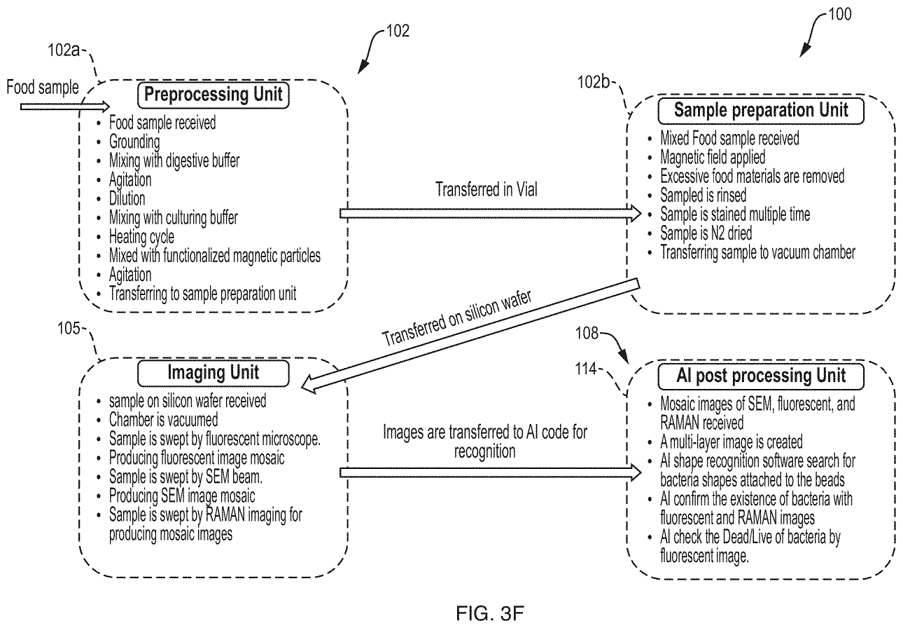

FIGS. 3A-3E provide a schematic depiction 200 various elements of an example of implementation of a method according to an embodiment for processing and analyzing a food sample under investigation to determine whether one or more pathogens of interest are present in the sample.

As illustrated in FIG. 3A, a substrate 200 can be placed over a magnet such as an electromagnet 202 and placed below an outlet 204 of a container 206 (e.g., as shown in steps 1 and 2, which can be performed in any order). As shown in the panel designated as (Detail A), a food sample 208 (e.g., after being crushed and/or emulsified) and a buffer 210 (e.g., a mixture of water and PBS) and optionally a digestive enzyme (e.g., an enzyme that can break down connective tissue) can be placed within a container 212 and mixed. In some cases, the mixture can be diluted and optionally a culturing buffer 214 can be added and the mixture subjected to a heating cycle to cause the growth of a pathogen of interest, when present in the sample. For example, the culturing step can be performed over a period of 20-30 minutes. Subsequently, a plurality of capture particles such as magnetic particles 216 functionalized with a molecular recognition element that exhibits specific binding to the pathogen of interest can be added to the container 212. The mixed or processed sample is then transferred to the container 206 that is sealed at its bottom end to a surface of the substrate 200 (step 3). This is followed by activating a quadrupole magnet 220 and the magnet 202 positioned below the substrate 200. The quadrupole magnet 220 can generate a magnetic field within the container 206 that facilitates the axial movement of the magnetic particles 216, which can be attached to a pathogen of interest, onto the surface of the substrate 200. The magnet 202 positioned below the substrate 200 can help retain the magnetic particles 216 on the substrate surface (step 4).

With particular reference to FIG. 3B, subsequently, the remaining liquid in the container 206 is discharged (step 5), the container 206 is removed from the substrate 200 (step 6), and a washing plate 222 is disposed over the surface of the substrate 200 (step 7). A washing liquid 224 (e.g., deionized water) can be transferred via capillary action provided by the washing plate 222 onto the substrate surface 200 (step 8).

With particular reference to FIG. 3C, the washing plate 222 can be detached from the substrate 200 and the washing liquid removed (step 9). This is followed by flowing an inert drying gas (e.g., N2 gas) 226 onto the substrate 200 (step 10). Subsequently, the washing plate 222 can be coupled to the surface of the substrate 200 (step 11) and a staining liquid 228 (e.g., a liquid containing fluorescent dyes for staining live and dead pathogen particles) is transferred via the washing plate 222 onto the substrate 200 surface (step 12). The staining liquid 228 can cause staining of pathogen particles, if any, that are attached to the magnetic particles 216.

With particular reference to FIG. 3D, the staining liquid 228 can be removed from the substrate 200 surface (step 13). This is followed by using the washing plate 222 to transfer washing liquid 230 onto the substrate 200 surface (step 14), followed by removing the washing liquid 230 and deactivating the magnet 202 (step 15). The steps 11-15 can be repeated as needed, e.g., between 5 and 10 times. Subsequently, the inert drying gas 226 (e.g., N2 gas) is passed over the substrate 200 surface in a drying step (step 16).

With particular reference to FIG. 3E, the flow of the inert drying gas 226 is terminated (step 17) and the substrate 200 is removed from the magnet 202 (step 18) for transfer to a vacuum chamber 230 of a combined fluorescent/SEM imaging system 105 (step 19).

The substrate 200 is then ready for fluorescent and SEM analysis. A fluorescent tile image of the substrate 200 can then be obtained and an SEM tile image of the substrate can be obtained. The images can be analyzed, e.g., in a manner discussed above, to identify pathogen particles of interest, if any, within those images (not shown). For example, an AI analysis system can be employed to process the fluorescent and SEM images in order to identify the pathogen particles, if any, within the images.

Further, the fluorescent and the SEM images can be correlated to attempt to generate a one-to-one correspondence between each pathogen particles identified in the fluorescent and SEM images.

FIG. 3F schematically depicts various modules of a system 100 according to an embodiment of the present teachings, which includes a sample preparation/processing module having a sample pre-processing unit 102a and a sample preparation unit 102b, an imaging system 105, and an AI post processing unit 114 of an analysis module 108. In this embodiment, each module is configured to perform the steps depicted in FIG. 3F. While in some embodiments, a fluorescence imaging system in combination with an SEM imaging system is employed for obtaining images of the pathogen particles, if any, attached to the magnetic particles, in other embodiments, a combination of Raman imaging system and an SEM imaging system is employed. Further, in some embodiment, the imaging unit can include a fluorescent image system, a Raman imaging system as well as an SEM imaging system.

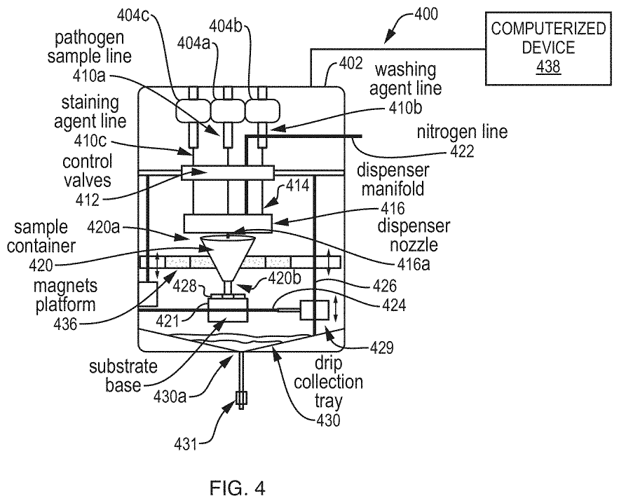

The sample processing/analysis according to the present teachings can be implemented using a variety of different designs. By way of example, FIG. 4 schematically depicts an example of a sample-processing device 400 according to an embodiment, which can be used in the practice of the present teachings. As discussed in more detail below, the device 400 provides a funnel-shaped transfer element 420, which is positioned above a substrate and which can receive a liquid sample (e.g., a liquid extract obtained via mixing a food sample with one or more processing liquids and blending the mixture followed by mixing the liquid extract with a plurality of ferromagnetic particles that are functionalized with a molecular recognition element, such as an antibody or an aptamer).

A magnetic field can be employed to cause movement of the ferromagnetic particles onto the substrate's surface. As discussed in more detail below, in this embodiment, a magnetic array surrounding the funnel-shaped transfer element 420 can generate a magnetic field that can force the functionalized ferromagnetic particles received in the funnel-shaped transfer element 420 via a proximal end thereof onto the substrate's surface via the distal end of the funnel-shaped transfer element. In some implementations, such as that described below, the magnetic array can be moved axially as it surround the funnel-shaped transfer element 420 to facilitate the transfer of the functionalized ferromagnetic particles onto the substrate's surface. Further, a magnet positioned below the substrate can help retain the ferromagnetic particles on the substrate's surface.

The device 400 further includes three reservoirs (herein also referred to as containers), two of which can store a washing liquid and a staining liquid for washing the substrate's surface and staining pathogen particles, and the third is configured for receiving a liquid sample. Further, the device 400 can include a gas line for receiving an inert drying gas, e.g., nitrogen, and directing the gas onto the substrate's surface to dry the ferromagnetic particles. This allows performing a washing, inert drying, staining cycle for staining the pathogen particles and removing excess residue from the substrate's surface to facilitate the detection of the pathogen particles, if any, via subsequent fluorescent and SEM imaging, as discussed in more detail below.

More specifically, the device 400 includes a housing 402, which houses various components of the device 400. By way of example, the housing 402 can be formed of a suitable polymeric material, e.g., acrylic polymers, and can further include windows to allow a user to observe operation of the device. In this embodiment, the device 400 includes a particle concentration subassembly, a wash-stain-dry (WSD) subassembly, and a control/drive subassembly, as discussed in more detail below.

More specifically, in this embodiment, the particle concentration subassembly includes a container 404a provided on the top of the device for loading a liquid sample, e.g., a liquid sample containing a mixture of functionalized ferromagnetic particles and a liquid extract of a food sample. Two reservoirs 404b and 404c are also provided for storing a wash liquid and a staining liquid. As discussed above, in this embodiment, the staining liquid can include a mixture of two fluorescent dyes, one of which can preferentially stain live pathogen particles and the other can preferentially stain dead pathogen particles.

In this embodiment, a plurality of pipes 410a, 410b, and 410c (herein collectively referred to as pipes 410) connect the reservoirs 404a, 404b, and 404c (herein collectively referred to as reservoirs 404) to a control manifold 412, which can be considered as part of the control subassembly, in which a plurality of control valves (not visible in the figures) are disposed for controlling the flow of the liquids contained in the reservoirs onto the substrate.

A plurality of pipes 414a, 414b, and 414c (herein collectively referred to as pipes 414) connect each of the control valves disposed in the control manifold to a liquid dispense manifold 416, which is in turn fluidly coupled via a dispersion nozzle 416a to a proximal end 420a of a glass funnel 420 so as to deliver a mixture of the sample, the fluorescent dyes and wash liquid (e.g., a mixture of water and PBS) to the glass funnel 420. The distal end 420b of the glass funnel 420 is configured for coupling to the surface of a substrate 421, as discussed below, so as to form a seal with that surface for transferring the liquid mixture (herein also referred to as the liquid sample) received via the dispenser manifold onto the substrate's surface.

The device 400 can further include a gas line 422 that can be coupled to a source of inert drying gas (e.g., nitrogen) for directing a flow of the drying gas onto the substrate's surface for drying the material (e.g., functionalized ferromagnetic particles) deposited on the substrate's surface.

With continued reference to FIG. 4, the device 400 includes a substrate holder 424 that is movably coupled to a pair of rails 426. The device 400 includes a substrate loading/unloading section 428, where a sample can be loaded onto or removed from the substrate holder 424. A variety of substrates can be used in the practice of the present teachings. By way of example, the substrate can be a semiconductor substrate, such as a silicon substrate, though other materials can also be used.

The device 400 further includes a linear actuator 429 for moving the substrate holder 424 along the rails 426 and hence the substrate between the substrate loading/unloading section and a position below the glass funnel 420. In some embodiments, the linear actuator 429 can be implemented in a variety of known techniques as informed by the present teachings. For example, the linear actuator 429 can include a rod and pinion system that can be actuated using a motor, though a variety of other mechanisms can also be employed.

Upon activation of the device, the linear actuator 428 can be activated to move the substrate holder 426 and the associated substrate 421 along the pair of rails 428 so as to position the substrate 421 below the glass funnel 420. Another linear actuator (not shown) can be used to move the substrate holder 426 and the associated substrate 421 along an axial direction so as to couple the distal end 420b of the glass funnel 430 to the substrate 421. For example a seal can be used to seal a portion of the substrate 421 loaded onto the substrate holder 426 when the substrate holder 426 is coupled to the distal end 420b of the glass funnel 420. After dispensing the liquid sample onto the sealed portion of the substrate surface the substrate holder 426 can be moved to provide a small gap between the distal end of the glass funnel 420 and the substrate 421 to allow excess liquid on the substrate surface to be delivered to a drip collection tray 430. In this embodiment, an opening 430a provided in the drip collection tray can be used to discharge the excess liquid collected in the drip collection tray via a valve 431.

With continued reference to FIG. 4, a magnetic array 432 comprising a plurality of magnets, e.g., a Halback array, is positioned around the glass funnel 420 and is movable along the axial direction via a linear actuator 434 to apply a substantially axial magnetic field to the ferromagnetic particles when the liquid sample is released from the dispenser manifold into the interior of the glass funnel 420 so as to force the ferromagnetic particles to move downward toward the substrate's surface, thereby facilitating the delivery of the ferromagnetic particles onto the substrate surface.

Further, a magnet 436, e.g., an electromagnet, is positioned below the substrate holder to help retain the ferromagnetic particles on the substrate surface once they are deposited on that surface.

The device can further include a computerized device 438 having a controller (e.g., a processor and memory), which can be implemented in hardware, software and/or firmware, disposed in electrical communication with the device 400 and configured to control the operation of the device 400.

In use, a user can fill the wash and stain reservoirs 404a, 404b with a wash liquid and a staining liquid (e.g., a liquid containing two fluorescent dyes as discussed above), respectively, and transfer a liquid sample of interest into the sample reservoir 404c. Further, the user can load a substrate 421 onto the substrate holder 426. The user can be then activate the device 400, e.g., via an activation switch. The programmed controller 438 can then cause the device 400 to move the substrate 421 below the glass funnel 420 and cause a sealing engagement of the substrate 421 with the distal end 420b of the glass funnel 420. Further, once the substrate 421 is coupled to the glass funnel 420, the controller 438 can activate the control valves 412 within the control manifold to allow the liquid sample and the staining liquid to be released from their respective reservoirs 404c, 404b and be transferred (e.g., under the influence of gravity) into the dispense manifold 416 and from the dispense manifold 416 onto the substrate surface.

The controller 438 can then initiate a wash, drying, stain (WDS) cycle by activating a valve to allow the flow of an inert drying gas (e.g., nitrogen) onto the substrate surface to dry the functionalized ferromagnetic particles, followed by activating the control valve associated with the wash reservoir 404b to allow the wash liquid to be introduced onto the sample in order to wash at least a portion of excess stain liquid. This can be followed by actuating the valve associated with the stain liquid reservoir 404c to introduce the stain liquid onto the substrate 421. In some embodiments, the controller 438 can be programmed to repeat the WDS cycle a predetermined number of times.

After completion of the WDS cycle, the controller 438 can again actuate the valve associated with the inert gas line 422 so as to introduce a flow of the gas onto the substrate 421 to ensure that the ferromagnetic particles are dried. The controller 438 can then turn off the magnet below the substrate holder and move the substrate holder 426 so as to transition the substrate to the substrate loading/unloading section, where a user can remove the substrate 421 from the device 400.

Following acquisition of fluorescent and SEM images of the ferromagnetic particles distributed over the substrate surface, a pathogen detection system 500 can be utilized for identifying pathogen particles, if any, that are attached to the functionalized ferromagnetic particles.

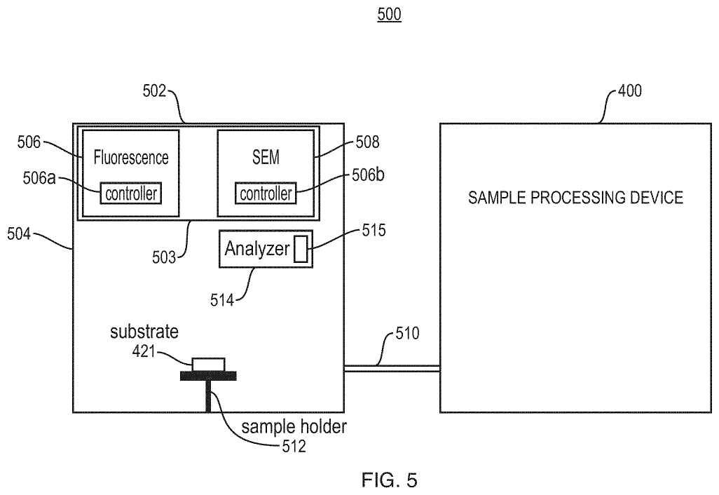

FIG. 5 is a schematic view of an embodiment of a pathogen detection system 500 according to an embodiment, which includes the above sample processing device 400 as well as a sample analysis module 502, which includes a combined fluorescent/SEM imaging device 503 housed in a vacuum chamber 504 to which a fluorescent imaging system 506 and an SEM imaging system 508 are coupled.

In this embodiment, the sample processing device 400 is coupled via a conduit 510 to the fluorescent/SEM imaging device 502 via which the substrate 421 prepared by the sample processing device 400 can be delivered automatically to a sample holder 512 provided in the fluorescent/SEM imaging device 502.

The fluorescent and SEM imaging systems 506, 508 can obtain fluorescent and SEM images of the substrate surface on which the ferromagnetic particles are distributed. In some cases, each of the SEM and the fluorescent imaging systems 506, 508 can include its own dedicated controller (e.g., controllers 506a and 506b schematically illustrated in FIG. 5 and each having a memory and a processor) that can control the operation of the imaging systems 506, 508, e.g., generating tile images of the substrate's surface. In other embodiments, the functionality for controlling the operation of the fluorescent and the SEM imaging systems can be integrated in a single controller unit.

The device 500 can further include an analysis module 514 (herein also referred to as an analyzer), such as a computerized device having a processor and memory, that is configured to receive the fluorescent and the SEM images generated by the fluorescent and SEM imaging systems 506, 508 and process those images to identify pathogen particles, if any, that are attached to the functionalized ferromagnetic particles. For example, the analysis module 514 can include an image processing unit that can analyze the fluorescent images to identify pathogen particles, if any, that have been stained with the fluorescent dyes. Further, the analysis module 514 can be configured to determine in which fluorescent channel (e.g., red or green) the fluorescent signals were detected to determine whether the fluorescent signals were emitted by stained live or dead bacteria.

In this embodiment, the analysis module 514 can include an artificial intelligence (AI) analysis system 515 that can analyze the SEM images to identify the pathogen particles, if any, that are attached to the ferromagnetic particles. In some cases, the AI system 515 can execute supervised AI algorithms, which have been trained on images of pathogens of interest, to identify the images of one or more pathogen particles of interest within the SEM image. Further, as discussed above, in some embodiments, the functionalized ferromagnetic particles can include multiple classes of particles with different sizes, where the particles within each class are functionalized with a different molecular recognition element (e.g., a different antibody) exhibiting specific binding to a different pathogen. In such cases, upon detection of a pathogen particle attached to a ferromagnetic particle, the analysis module 514 can rely on the relative size (e.g., diameter) of the particle to identify the type of pathogen (e.g., whether the pathogen particle is an E. coli or a Salmonella bacterium).

The analysis module 514 can also be configured to correlate an SEM and respective fluorescent image to determine whether a pathogen particle identified in one image is present in the other image. In some embodiments, the substrate surface on which the magnetic particles are deposited can include one or more markers, e.g., in the form of one or more grooves etched onto the substrate surface, which can facilitate correlating the fluorescent and the SEM images.

The analysis module 514 can then determine, based on all of the available data, whether a pathogen of interest was present in the food sample under investigation.

In some embodiments, rather having data processing capability on board of the device 500, the device 500 can include a communication module (not shown in this figure) for transferring data corresponding to the fluorescent and SEM images to a remote server, where the data can be processed to identify one or more pathogen particles of interest, if any, in the images.

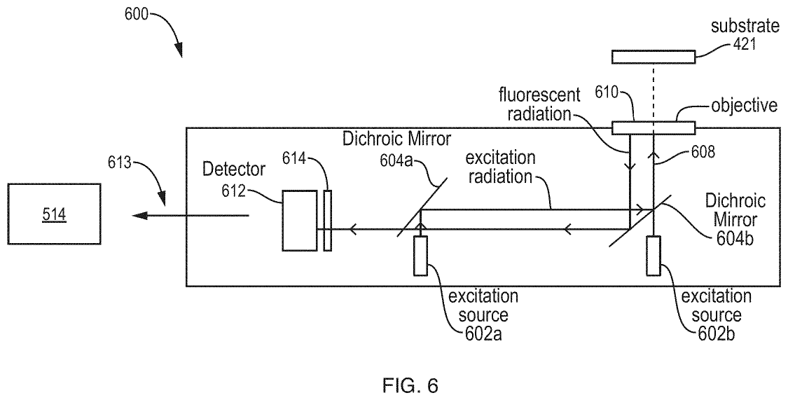

A variety of fluorescent and SEM imaging systems can be employed in the practice of the present teachings. For example, FIG. 6 schematically depicts an example of a fluorescent imaging system 600 that can be employed in the practice of the present teachings. The fluorescent imaging system 600 includes two excitation light sources 602a/602b (e.g., LEDs or diode lasers), one of which generates excitation radiation for exciting one of the fluorescent dyes utilized in processing a sample and the other generates excitation radiation for exciting the other fluorescent dye. In some embodiments, a single broadband source with appropriate filters can be utilized to generate radiation for exciting both fluorescent dyes. In this embodiment, two dichroic mirrors 604a/604b direct the excitation light generated by the two light sources 602a/602b, respectively, onto a common path 608. An objective 610 focuses the excitation radiation onto a sample under investigation, which is in this case includes the functionalized magnetic particles distributed on the surface of the substrate 421.

The radiation emitted by the fluorescent dyes staining the pathogen particles, if any, that are coupled to the magnetic particles is directed via reflection by the dichroic mirror 604b and passed through the dichroic mirror 604a toward a detector 612. A filter 614 positioned in front of the detector 612 mitigates or prevents the passage of excitation wavelengths but allows the fluorescent wavelengths to pass onto the detector 612. The detector 612 is configured to generate fluorescent detection signals in response to the detection of the incident fluorescent radiation. The fluorescent detection signals 613 generated by the detector 612 can be received by the above analyzer 514, which is configured to analyze the fluorescent detection signals 613 in a manner discussed herein to identify images of live and/or dead pathogen particles, if any, the image. Further details regarding a fluorescent imaging system suitable for use in the practice of various embodiments as modified based on the present teachings is disclosed in U.S. Published Patent Application No. 2021/0325309 titled “Optical Module With Three Or More Color Fluorescent Light Sources And Methods For Use Thereof,” which is herein incorporated by reference in its entirety.

With reference again to FIG. 5, in this embodiment, the substrate holder 512 is movable along the x- and y-dimensions to allow acquiring a fluorescent tile image of the substrate surface.

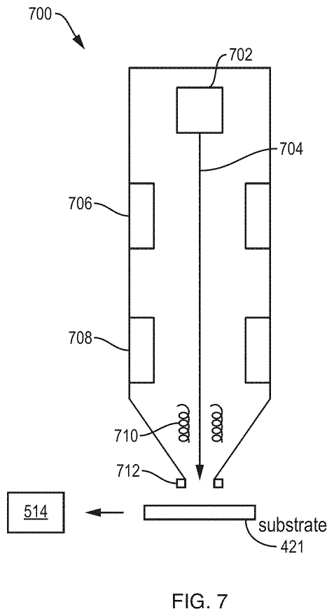

FIG. 7 schematically depicts an SEM imaging system 700 suitable for use in various embodiments of the present teachings. The SEM imaging system 700 includes an electron gun 702 that generates an electron beam 704. A condenser lens 706 and a focusing lens 708 cooperatively concentrate the electron beam generated by the electron gun onto a small portion of the surface of the substrate 421 mounted onto the substrate holder 512. A scanning coil 710 can scan the electron beam over substrate surface. A detector 712 can detect back-scattered and/or secondary electrons emanating from the portions of the substrate that are exposed to the electron beam to generate a plurality of electron detection signals. The above analyzer 514 can receive the electron detection signals and can process those signals to generate an SEM tile image of the substrate surface and in particular that of the functionalized magnetic particles and pathogen particles, if any, attached to one or more of the magnetic particles. In some embodiments, the teachings of U.S. Pat. No. 11,183,362 titled “Scanning electron microscope apparatus and operation method thereof,” which is incorporated herein by reference, as informed by the present teachings can be employed in the practice of various embodiments of the present teachings.

In some embodiments, the substrate holder 512 can be tilted about the x- and/or y-dimension and SEM images of the substrate surface at different angles can be acquired and analyzed to enhance the probability of detecting pathogen particles attached to the functionalized magnetic particles.

In some embodiments, in a microfluidic device may be utilized to allow the fluorescently-tagged pathogen particles to interact with the functionalized magnetic particles.

In another aspect, a method of detecting one or more pathogens in a sample is disclosed, which includes processing a first portion of the sample in a manner disclosed herein via staining at least one type of pathogen particles, if any, with a fluorescent dye and concentrating the stained pathogen particles, if any, using a plurality of functionalized beads, such as magnetic beads that exhibit specific binding to the target pathogen. Another portion of the sample (herein referred to as the second portion) can be incubated with a culture medium at an appropriate temperature to allow growth of the target pathogen, if present in the sample. The incubation period can be adjusted, e.g., based on time constraint, the sensitivity of the fluorescent interrogation system, among others. By way of example, and without limitation, in some cases, the incubation period can be in a range of about 1 hour to about 10 hours, e.g., in a range of about 2 hours to about 8 hours, or in a range of about 3 hours to about 5, hours.

In some such embodiments, while the second sample is being incubated with the growth media, the beads associated with the first sample can then be interrogated via excitation of the fluorescent dye and detecting the emitted fluorescent radiation.

Subsequently, following the completion of the incubation period, the second sample is processed via staining with the fluorescent dye and incubated with the functionalized beads to capture the target pathogen, if present in the sample. The beads are then interrogated using a fluorescence excitation and detection. The fluorescent signal, if any, obtained via interrogation of the first sample is compared with the fluorescent signal obtained via interrogation of the second sample to determine whether the target pathogen is present in the sample, and if so, whether the target pathogen is dead or alive.

The detection and comparison of the fluorescent radiation from the two samples (i.e., the first and the second samples) may lead to the following situations: (1) fluorescent signals are detected for both samples and the intensity of the fluorescent signal associated with the second sample is greater than the intensity of the fluorescent signal associated with the first sample, (2) fluorescent signals are detected for both samples and the intensities of the detected fluorescent signals are substantially the same, (3) No fluorescent signal within the limit-of-detection of the fluorescent excitation/detection is detected for the first sample, but it is detected for the sample, (4) fluorescent signal is detected for the first sample but no fluorescent signal within the limit-of-detection of the fluorescent excitation/detection system is detected for the second sample, and (5) fluorescent signals are detected for both samples but the fluorescent signal for the second sample is less than the fluorescent signal for the first sample.

In case (1), one may conclude that live particles of the target pathogen are present in the sample. In case (2), one may conclude that one cannot determine conclusively whether live particles of the target pathogen are present in the sample. In case (3), one may conclude that the concentration of the live particles of the pathogen in the first sample was not sufficient to allow detection while the growth of the target pathogen in the second sample resulted in a sufficient number of live particles of the pathogen to allow their detection. In case (4), one may conclude that the result are inconclusive and the analysis should be repeated.

In some embodiments, only fluorescence detection or SEM detection may be sufficient for determining whether a target pathogen is present in a sample at a level above a predefined threshold. As such, various embodiments of the present teachings may be practiced using only one of those modalities, i.e., only using fluorescent detection or SEM.

The following examples are provided to further illustrate various aspects of the present teachings and are not presented to necessarily indicate optimal ways for practicing the present teachings and/or optimal results that can be obtained.

### EXAMPLES

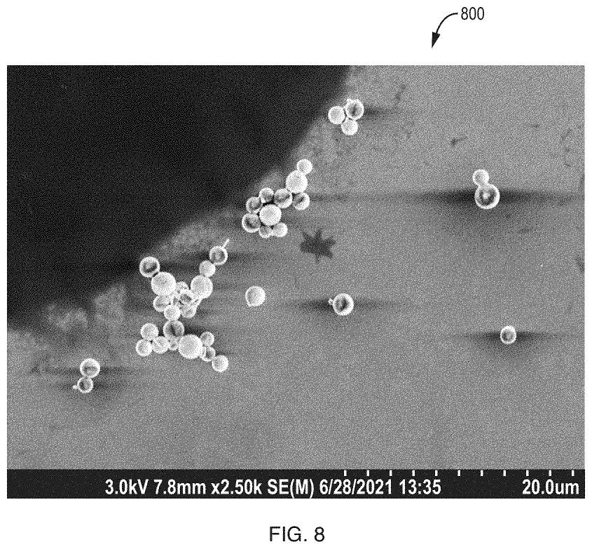

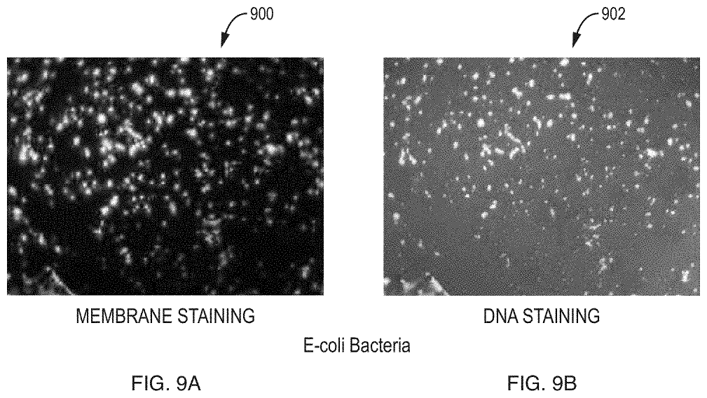

E. coli bacteria were cultured and were attached to ferromagnetic particles that were functionalized with an antibody exhibiting specific binding to E. coli. FIG. 8 shows an SEM image 800 of such pathogen-coupled ferromagnetic particles in which a several E. coli bacteria are visible. Another batch of E. coli bacteria was cultured. A portion of the cultured bacteria was heat killed and another portion was fixated to substrate without being subjected to a heat killing protocol. The E. coli bacteria were then attached to ferromagnetic particles functionalized with anti-E. coli antibody. The ferromagnetic particles were then exposed to membrane-staining and DNA-staining fluorescent dyes. The DNA-staining dye can stain the heat-killed bacteria and not the fixated bacteria while the membrane-staining dye can stain both types of bacteria. FIGS. 9A and 9B show fluorescent images 900, 902, respectively, of the stained bacteria illustrating that the color of the detected fluorescent radiation can be used to distinguish between the two types of bacteria, thereby indicating that the methods of the present teachings can be used to distinguish between dead and live bacteria in a sample, e.g., a food sample.

In a series of experiments, polyclonal E. coli antibodies from Bio-Rad or Novus Biologicals were conjugated to magnetic Dynabeads (ThermoFisher Scientific) with either surface protein A or epoxy groups. E. coli bacteria were grown overnight in LB media (e.g., Lysogeny broth) and then phosphate buffered solution (PBS) washed and resuspended in PBS with either 0%, 0.02%, or 0.05% Triton X-100 detergent to a concentration of 10⁹ CFU/ml. The bacteria were then 10-fold serially diluted 6 times. Several 90 μl aliquots of the 10⁸ CFU/ml dilution were incubated with either 10 μl of one of the antibody-bound beads, or a Dynabead control without antibodies, for 2 hours at either 37° C. or 42° C. Subsequently, a magnetic stand was used for pulldown and the beads were gently washed with PBS twice before being resuspended in 27 μl of PBS. A volume of 27 μl of each bacterial dilution was also added to new tubes. A volume of 3 μl of Syto 9 live bacteria dye was added to both the bead suspensions and bacterial dilutions and was gently pipette mixed. The bead suspensions and diluted bacterial solutions were transferred to glass bottom plates and fluorescence was measured via excitation at 465 nm and emission at 515 nm. A standard curve was produced using the bacteria dilutions to determine approximately the actual bacteria pulldown in the bead samples.

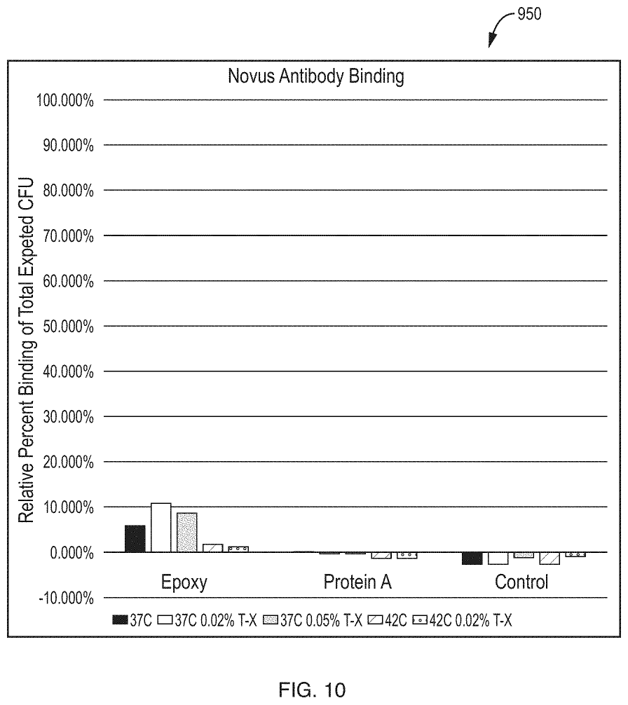

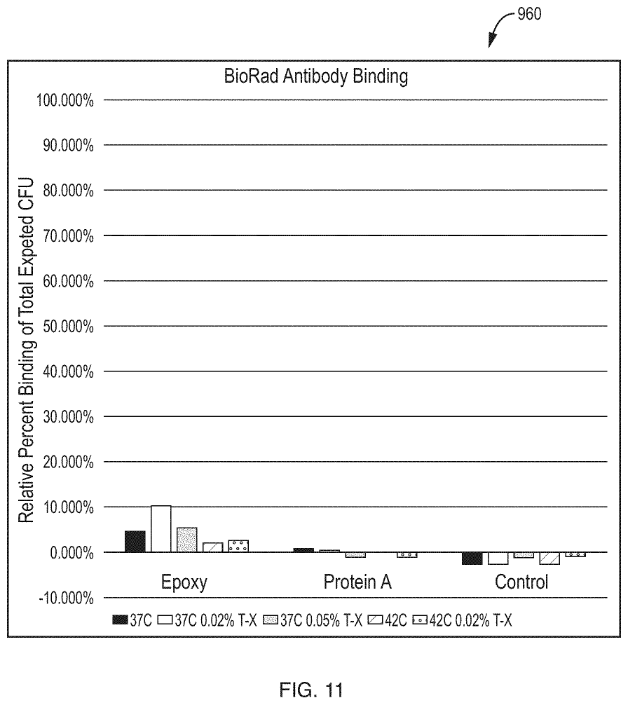

FIG. 10 is a graph 950 illustrating the results of binding of E. coli bacteria to the beads functionalized with antibodies obtained from Novus Biologicals and FIG. 11 is a graph 960 illustrating the results of the binding of E. coli bacteria to the beads functionalized with antibodies obtained from Bio-Rad. It was detected that the use of a small amount of detergent enhances the binding of the bacteria to the antibody-bound beads.

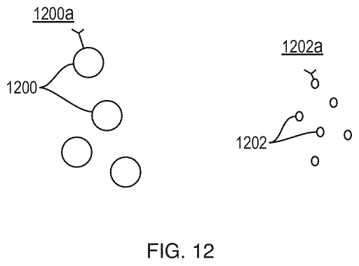

As noted above, in some embodiments, antibody-functionalized capture particles having different sizes can be utilized to distinguish one type of pathogen from another in an SEM image. By way of example, FIG. 12 schematically depicts two types of capture particles 1200 and 1202 having different sizes, where the capture particles 1200 are functionalized with antibodies 1200a that provide specific binding to E. coli bacteria and the capture particles 1202 are functionalized with antibodies 1202a that provide specific binding to Salmonella bacteria. As the sizes of the capture particles 1200 are different than those of capture particles 1202, the pathogens, if any, observed in an SEM image as coupled to the capture particles 1200 can be identified as E. coli bacteria and those coupled to the capture particles 1202 can be identified as Salmonella bacteria.

While various embodiments of the innovation have been particularly shown and described, it will be understood by those skilled in the art that various changes in form and details may be made therein without departing from the spirit and scope of the innovation as defined by the appended claims.

What is claimed is:

1. A method of detecting a pathogen in a sample, comprising:
mixing at least a portion of the sample with a plurality of capture particles functionalized with a molecular recognition element exhibiting specific binding to said pathogen so as to capture at least a portion of pathogen particles when present in the sample,
exposing said captured pathogen particles to at least two fluorescent dyes, which emit fluorescent radiation at two different wavelengths in response to excitation, such that live pathogen particles, if any, among said captured pathogen particles are preferentially stained with one of the dyes and dead pathogen particles, if any, among said captured pathogen particles are preferentially stained with the other dye,
irradiating the captured stained pathogen particles with excitation radiation to excite said fluorescent dyes,
detecting fluorescent radiation emitted by said excited fluorescent dyes, and
distinguishing said live pathogen particles from said dead pathogen particles based on wavelengths of the detected fluorescent radiation.

2. The method of claim 1, wherein said sample comprises a food sample.

3. The method of claim 1, wherein said pathogen comprises a food-borne pathogen.

4. The method of claim 3, wherein said pathogen comprises any of E. Coli, Salmonella, and Listeria.

5. The method of claim 1, wherein said capture particles comprise magnetic particles functionalized with said molecular recognition element.

6. The method of claim 5, further comprising applying a magnetic field to said magnetic particles after said mixing of the sample with the magnetic particles so as to concentrate said magnetic particles coupled to the pathogen particles, if any.

7. The method of claim 6, wherein said step of irradiating said captured stained pathogen particles with excitation radiation is performed subsequent to said step of applying the magnetic field to cause concentration of the magnetic particles coupled to said pathogen particles.

8. The method of claim 1, further comprising introducing said magnetic particles coupled to said pathogen particles into a scanning electron microscope (SEM) and obtaining an SEM image of at least a portion of said magnetic particles.

9. The method of claim 8, further comprising analyzing said SEM image to identify one or more pathogens, if any, coupled to said imaged magnetic particles.

10. The method of claim 9, further comprising using said detected fluorescent radiation to generate a fluorescent image of said pathogen particles coupled to said magnetic particles.

11. The method of claim 10, further comprising correlating said SEM image with said fluorescent image to correlate the fluorescent and SEM images of each of a plurality of magnetic particles identified in said SEM and fluorescent images.

12. A system for detecting at least one pathogen in a sample, comprising:
a substrate,
a plurality of capture particles each functionalized with at least one molecular recognition element for specifically binding to said at least one pathogen, said plurality of capture particles being configured for distribution over a surface of said substrate,
an electron microscopy system having a holder for receiving said substrate, said electron microscopy system being configured for generating one more electron microscopy images for said plurality of particles disposed on said substrate surface, and
an artificial intelligence system for receiving said one or more electron microscopy images and analyzing said images for detection of one or more of said at least one pathogen when bound to at least one of said plurality of capture particles.

13. The system of claim 12, wherein said capture particles comprise a plurality of magnetic particles.

14. The system of claim 12, further comprising a magnet for facilitating distribution of said plurality of the magnetic particles onto said substrate surface.

15. The system of claim 12, wherein said magnet comprises an electromagnet.

16. The system of claim 12, wherein said substrate holder is configured to be movable relative to an electron beam of said electron microscopy system to allow acquisition of said one or more electron images from different viewing angles.

17. The system of claim 12, wherein said magnetic particles have a maximum size less than about 2 micrometers.

18. The system of claim 12, wherein said molecular recognition element comprises an antibody exhibiting specific binding to said at least one pathogen.

19. The system of claim 13, wherein said plurality of capture particles comprises a first set of particles having a first size and a second set of particles having a second size, wherein said first set of the particles is functionalized with a first molecular recognition element exhibiting specific binding to a first pathogen and said second set of the particles is functionalized with a second molecular recognition element exhibiting specific binding a second different pathogen.

20. The system of claim 12, further comprising at least one processing reagent for processing said sample prior to introduction of said sample onto said functionalized capture particles.

21. The system of claim 12, wherein said sample comprises a food sample.

22. A method of detecting at least one pathogen in a sample, comprising:
processing a sample with one or more processing reagents to generate a processed sample,
mixing said processed sample with a plurality of capture particles functionalized with at least one molecular recognition element exhibiting specific binding to said at least one pathogen,
generating one or more electron microscopy images of said plurality of the particles, and
using an artificial intelligence system to identify said pathogen in said one or more microscopy images, when present in the sample.

23. The method of claim 22, further comprising transferring, subsequent to said mixing step, at least a portion of said plurality of the capture particles onto a surface of a substrate.

24. The method of claim 22, further comprising introducing said substrate into said electron microscopy system.

25. The method of claim 22, wherein said plurality of capture particles comprise magnetic particles.

26. The method of claim 22, further comprising using a magnetic field to facilitate introduction of said magnetic particles onto a surface of said substrate.

27. The method of claim 22, wherein said sample comprises a food sample.

28. The method of claim 1, further comprising suspending the sample in a solution having a detergent concentration.
